(19) United States
(12) Patent Application Publication
(10) Pub. No.: US 2002/0077358 A1
(43) Pub. Date: Jun. 20, 2002

(54) Use of hydroperoxyeicosatetraenoic acid derivatives following refractive surgery

(75) Inventors: John M. Yanni, Burleson, TX (US); Daniel A. Gamache, Arlington, TX (US); Steven T. Miller, Arlington, TX (US)

Correspondence Address: Alcon Universal Ltd., c/o Alcon Research, Ltd., R&D Counsel (Q-148), 6201 So. Freeway, Fort Worth, TX, 76134-2099, US

(73) Assignee: Alcon Universal Ltd.

(21) Appl. No.: 09/965,506

(22) Filed: Sep. 27, 2001

Related U.S. Application Data
(60) Provisional application No. 60/242,642, filed on Oct. 23, 2000.

(51) Int. Cl. A61K 31/22; A61K 31/20; A61K 31/16

(52) U.S. Cl. 514/549; 514/560; 514/626; 514/627

(57) ABSTRACT

The use of HETE derivatives to treat dry eye conditions associated with refractive surgery is disclosed.

This application claims priority to U.S. Provisional Application, U.S. Serial No. 60/242,642, filed Oct. 23, 2000.

The present invention is directed to the use of certain arachidonic acid metabolites to treat dry eye-type conditions associated with refractive surgery.

### BACKGROUND OF THE INVENTION

Dry eye, also known generically as keratoconjunctivitis sicca, is a common ophthalmological disorder affecting millions of Americans each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid manifest dry eye complications.

Although it appears that dry eye may result from a number of unrelated pathogenic causes, all presentations of the complication share a common effect, that is the breakdown of the pre-ocular tear film, which results in dehydration of the exposed outer surface and many of symptoms outlined above (Lemp, Report of the Nation Eye Institute/Industry Workshop on Clinical Trials in Dry Eyes, The CLAO Journal, volume 21, number 4, pages 221-231 (1995))

U.S. Pat. No. 5,696,166 (Yanni et al.) discloses compositions containing naturally occurring HETEs, or derivatives thereof, and methods of use for treating dry eye. Yanni et al. discovered that compositions comprising HETEs increase ocular mucin secretion when administered to a patient and are thus useful in treating dry eye. Although the '166 patent discloses the use of HETEs to treat dry eye conditions, it does not mention refractive surgery.

The number of refractive surgeries is increasing as the procedure known as laser in situ keratomileusis (LASIK) continues to gain acceptance. Other forms of refractive surgeries exist, such as radial keratotomy (RK) and photorefractive keratectomy (PRK). Dry eye-type symptoms, such as burning, stinging, foreign-body sensation and photophobia, can occur following refractive surgeries, especially LASIK procedures. Such dry eye-type symptoms can last from a few weeks to six months or more following surgery and are referred to herein as “transient dry eye conditions associated with refractive surgery.”

The exact cause of transient dry eye conditions associated with refractive surgery is not known but the procedure can either induce or exacerbate symptoms. LASIK may increase the severity of dry eye symptoms in patients with a pre-existing condition. Also, LASIK procedures sever sensory nerve endings in the creation of a corneal flap, which can impair the blink response. Reduction in blinking and reflex tearing can cause the cornea to dry out. In addition, the tear film can be negatively affected by local anesthetics during surgery as well as antibiotics and NSAIDs used post-surgically. Transient dry eye conditions associated with refractive surgery are currently treated using artificial tear or lubricating products in most cases and punctal plugs in severe cases.

### SUMMARY OF THE INVENTION

The present invention is directed to methods for the treatment of transient dry eye conditions associated with refractive surgery. The methods comprise the topical ophthalmic administration of a HETE derivative.

### DETAILED DESCRIPTION OF THE INVENTION

As used herein, the term “HETE derivative” refers to hydroxyeicosatetraenoic acid derivatives that stimulate mucin production and/or secretion in the conjunctival epithelium and goblet cells when topically applied, and are of the following formulas (I), (II) or (III):

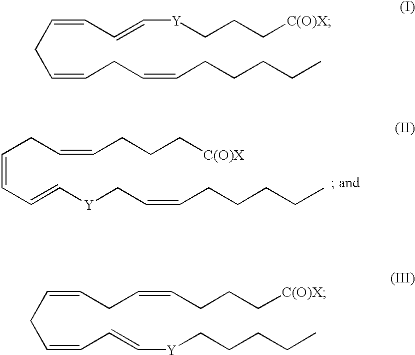

wherein:

X is OR or NHR′;

R is H, a cationic pharmaceutically acceptable salt moiety, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy;

R′ is H, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy; and

Y is

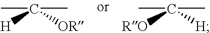

wherein R″ is H or C(O)R′.

Preferred compounds of the present invention include:

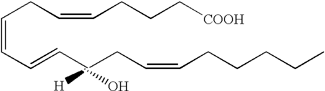

5,8,11,1 3-Eicosatetraenoic acid, 1 2-hydroxy-, [S-(E,Z,Z,Z)]-(“12(S)-HETE”) and

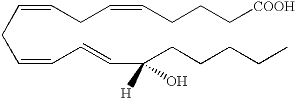

5,8,10,14-Eicosatetraenoic acid, 1 5-hydroxy-, [S-(E,Z,Z,Z)]-(“15(S)-HETE”).

15(S)-HETE is a the most preferred HETE derivative of the present invention.

The stereochemistry of the HETE derivatives of the present invention is important. Arachidonic acid occurs naturally as a 20-carbon, 4-double bond molecule. The double bonds are all cis at carbon positions of 5, 8, 11 and 14. Similarly, naturally occurring HETE derivatives resulting from arachidonic acid oxidation, generally contain 4 double bonds between particular carbons and in particular conformations (i.e., cis or trans). As described above, the HETE derivatives of the present invention have double bond conformations of 5,8,11 cis, 13 trans for 15-HETE; 5,8,14 cis, 10 trans for 12-HETE; and, 6 trans, 8,11,14 cis for 5-HETE. As further described above, the hydroxy group can be in the “R” or “S” conformation. Racemic mixtures of HETE derivatives containing R and S hydroxy derivatives at the 5, 12 and 15 positions, respectively, are also contemplated by the present invention.

The HETE derivatives of the present invention are further described in U.S. Pat. No. 5,696,166, the entire contents of which are hereby incorporated by reference.

The HETE derivatives of the present invention may be contained in various types of pharmaceutical compositions, in accordance with formulation techniques known to those skilled in the art. In general, the HETE derivatives will be formulated in solutions for topical ophthalmic administration. Aqueous solutions are generally preferred, based on ease of formulation, biological compatibility, as well as a patient's ability to easily administer such compositions by means of instilling one to two drops of the solutions in the affected eyes. However, the HETE derivatives may also be readily incorporated into other types of compositions, such as suspensions, viscous or semi-viscous gels or other types of solid or semi-solid compositions. Suspensions may be preferred for HETE derivatives that are not soluble in water at target concentrations. The ophthalmic compositions of the present invention may also include various other ingredients, such as buffers, preservatives, co-solvents and viscosity building agents.

Antioxidants may be added to compositions of the present invention to protect the HETE derivatives from oxidation during storage. Examples of such antioxidants include vitamin E and analogs thereof, ascorbic acid and butylated hydroxytoluene (BHT). Ophthalmic products are typically packaged in multidose form. Preservatives are thus required to prevent microbial contamination during use. A preferred preservative is polyquaternium-1. Such preservatives are typically employed at a level of from 0.001 to 1.0% weight/volume (“% w/v”).

Ethanol is optionally included in the compositions of the present invention at a concentration that enhances the biological efficacy of the HETE derivative. The mechanism by which ethanol appears to enhance the efficacy of the HETE derivatives of the present invention is not known; ethanol may not enhance the efficacy of all HETE derivatives. If present, the concentration of HETE derivative will range from about 0.001-2% w/v. Compositions containing HETE derivative concentrations of about 0.00001-0.02% w/v preferably will contain ethanol in a concentration of about 0.005-0.2% w/v, and most preferably, about 0.02-0.10% w/v.

Preferably, the compositions administered according to the present invention will contain a surfactant. Various surfactants useful in topical ophthalmic formulations may be employed. The surfactant(s) may provide additional chemical stabilization of the HETE derivatives and may further provide for the physical stability of the compounds. In other words, the surfactants may aid in preventing chemical degradation of the HETE derivatives and also prevent the HETE derivatives from binding to the containers in which their compositions are packaged. As used herein, “an effective concentration of surfactant(s)” refers to a concentration that enhances the chemical and physical stability of formula (I) compound(s). Examples of suitable surfactants include, but are not limited to: Cremophor® EL, polyoxyl 20 ceto stearyl ether, polyoxyl 40 hydrogenated castor oil, polyoxyl 23 lauryl ether and poloxamer 407 may be used in the compositions. A preferred surfactant is polyoxyl 40 stearate. The concentration of surfactant will vary, depending on the concentration of HETE derivative and ethanol, if any, present in the formulation. In general, however, the surfactant(s) concentration will be about 0.001 to 2.0% w/v. Preferred compositions of the present invention will contain about 0.1% w/v of polyoxyl 40 stearate.

In general, the topical composition applied to treat transient dry eye conditions associated with refractive surgery will contain one or more HETE derivatives in a total concentration range of from 0.001 to about 1.0% w/v. The dosing regimen will vary among patients at the discretion of the physician, but will typically be 1-2 drops administered 1-4 times per day.

The following example is presented to illustrate various aspects of the present invention, but is not intended to limit the scope of the invention in any respect.

### EXAMPLE 1

| Ingredient | Amount (% w/v) |
|---|---|
| HETE derivative | 0.00001-0.01 |
| Ethanol | 0-0.0505 |
| Polyoxyl 40 Stearate | 0.1 |
| Boric Acid | 0.25 |
| Sodium Chloride | 0.75 |
| Disodium Edetate | 0.01 |
| Polyquaternium-1 | 0.001 |
| NaOH/HCl | q.s., pH = 6.5-7.8 |
| Purified Water | q.s. 100% |

The above composition is prepared by the following method. The batch quantities of polyoxyl 40 stearate, boric acid, sodium chloride, disodium edetate, and polyquaternium-1 are weighed and dissolved by stirring in 90% of the batch quantity of purified water. The pH is adjusted to 7.5±0.1 with NaOH and/or HCl. Under yellow light or reduced lighting, the batch quantity of HETE derivative as a stock solution in ethanol and the additional quantity of ethanol necessary for the batch are measured and added. Purified water is added to q.s. to 100%. The mixture is stirred for five minutes to homogenize and then filtered through a sterilizing filter membrane into a sterile recipient.

Preferably, the above process is performed using glass, plastic or other non-metallic containers or containers lined with such materials.

The invention in its broader aspects is not limited to the specific details shown and described above. Departures may be made from such details within the scope of the accompanying claims without departing from the principles of the invention and without sacrificing its advantages.

What is claimed is:

1. A method for the treatment of transient dry eye conditions associated with refractive surgery comprising administering a composition comprising a pharmaceutically acceptable carrier and a pharmaceutically effective amount of one or more HETE derivative according to formulas (I), (II) or (III): 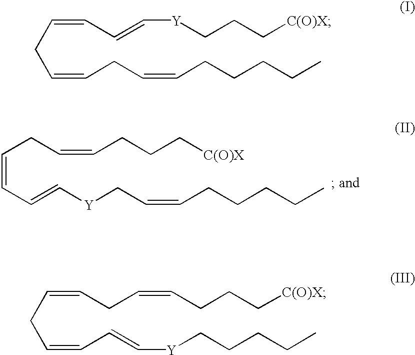
wherein:
X is OR or NHR′;
R is H, a cationic pharmaceutically acceptable salt moiety, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy;
R′ is H, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy; and
Y is 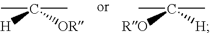
wherein R″ is H or C(O)R′.

2. The method of claim 1 wherein the HETE derivative is selected from the group consisting of 5(S)-HETE, 5(R)-HETE, 12(S)-HETE, 12(R)-HETE, 15(S)-HETE, 15(R)-HETE and racemates thereof.

3. The method of claim 2 wherein the HETE is 15(S)-HETE.

4. The method of claim 2 wherein the HETE is 12(S)-HETE.

5. The method of claim 1 wherein the composition is topically administered.

6. The method of claim 1 wherein the transient dry eye conditions associated with refractive surgery are associated with laser in situ keratomileusis.

7. The method of claim 1 wherein the transient dry eye conditions associated with refractive surgery comprises diffuse lamellar keratitis.
